(19) United States
(12) Patent Application Publication
(10) Pub. No.: US 2016/0095575 A1
(43) Pub. Date: Apr. 7, 2016

(54) METHOD AND DEVICE FOR QUANTITATIVE DYNAMIC EVALUATION OF SKELETAL MUSCLES FUNCTIONALITY

(71) Applicants: AMID S.R.L., Sulmona , AQ (IT); FONDAZIONE APOSTOLO, Milano (IT)

(72) Inventors: Laura Bertele, Merate (IT); Carlo Apostolo, Merate (IT); Gianni Pedrizzetti, Prato (IT); Giovanni Tonti, Sulmona (IT)

(21) Appl. No.: 14/786,571

(22) PCT Filed: Feb. 13, 2014
(86) PCT No.: PCT/IB2014/058966

§ 371 Date: Oct. 23, 2015

(30) Foreign Application Priority Data
Apr. 23, 2013 (IT) AQ2013A000003

(51) Int. Cl. A61B 8/00; A61B 8/14; A61B 8/08; G06T 7/00; G06T 7/20

(52) U.S. Cl. CPC A61B 8/4227; G06T 7/0016; G06T 7/2066; A61B 8/08; A61B 8/14; A61B 8/4477; A61B 8/4236; G06T 2207/30004

(57) ABSTRACT

The invention relates to a method for quantitative dynamic evaluation of skeletal muscles functionality comprising the following steps: a) receiving one or more sequences of two-dimensional or three- dimensional echographic images of the muscle under investigation; b) transforming such sequence or sequences of images in sequences of measurements of deformations and/or strain rates in more spatial locations of the muscle or the muscles to evaluate; c) outputting such sequences of spatial measurements in numeric and/or graphical format. A corresponding device is also disclosed.

The invention relates to a method and a device for quantitative dynamic evaluation of skeletal muscles functionality.

The performance of skeletal muscles depends on the ability of the same to develop a force and, for that reason, training is generally focused on muscle mass development. It is also true that the quality of muscle's action depends on many additional factors that are rarely assessed.

The first element involved in the quality of the development of strength concerns with the uniformity of the distribution of muscle tension along the anatomical structure, from bone insertion, through the tendons and along the muscle itself. It is thus important to be able to verify, from a dynamic point of view, the affective direction of stress and traction on the insertion points and how the muscle shortens according to its contractile ability. A muscle that presents, for example, a reinforced region connected to a less powerful one causes a local increase of the stresses in the areas where the region connects, with a corresponding increase of the potential risk of injury. These non-uniformities in the muscular function may develop as a result of a rehabilitation program or an athletic intense workload or for individual physiological characteristics of the athlete.

A second element of fundamental importance for the performance of the musculature relates to the presence or not of an adequate dynamic balance between the various muscular structures. Each action of the musculoskeletal system is driven by a number of muscles (agonists) that work together to perform the action in the most appropriate manner with the desired strength in the required time and along the correct direction in space and a corresponding number of muscles (antagonists) that are released so as not to oppose to the motion. This is what happens in every joint, the flexion of the hips, knees, ankles, as well as in the movements of the arms, hands and feet, even up to the toes, lumbar and cervical spine to mention only the main ones, or the opening and closing of the jaw.

In simplified terms, every action performed by a muscle element or elements (the agonists) involves a reaction of another element or elements (antagonists): the contraction of a muscle agonist is necessarily accompanied by the corresponding relaxation of the antagonist muscle. It is therefore evident the necessity of a balanced development between agonist and antagonist. A very common example in football, caused by normal training techniques, is the imbalance between the quadriceps (agonist of knee extension) and the flexors (antagonist). Excessively enhanced flexors restrain the action of the quadriceps and cause inaccuracy in the shooting besides causing overload on the knee with subsequent risk of muscle and tendon injuries.

It is therefore necessary to develop methods of training and preparation in football and in other disciplines, such as dance, as well as in therapeutic pathways including orthodontic ones, allowing a balanced development of the muscles. This balance is the essential objective in training and rehabilitation techniques.

The difficulties in the development of such balanced muscle training methods are mainly of a technical nature due to the lack of technology, simple and non-invasive, for a reproducible and objective measurement of the muscular capacity dynamics, i.e. during a muscular action, involving a single or multiple muscle elements at the same time. This technological deficiency has not allowed the development of case studies thus leaving the quality of the training completely in the hands of the individual trainer.

From document EP1297786 it is known how to use echographic acquisitions to evaluate the anatomic properties of muscle structures. This document goes in the right direction as it outlines how a simple and non-invasive methodology as the echographic one can be used to quantitative study muscle functionality. This study, however, is limited to the evaluation of the dilation and contraction in a region of a muscle by using spatio-temporal diagrams that provide the entity of the deformation along the longitudinal direction of the muscle. This allows measuring changes in the contraction of a muscle, for example as a function of different training sets, but it doesn't allow evaluating the uniformity of the muscle activity, as well as the synchronicity of the deformation between agonist and antagonist muscles, which represent indispensable aspects for the development of balanced training methodologies.

It is thus an object of the present invention to overcome, at least partially, these drawbacks.

The invention reaches the aim with a method for quantitative dynamic evaluation of skeletal muscles functionality comprising the following steps:
a) receiving one or more sequences of two-dimensional or three-dimensional echographic images of the muscle under investigation, particularly sequences of images related to different muscles and/or different zones of the same muscle;
b) transforming such sequence or sequences of images in sequences of measurements of deformations and/or strain rates in more spatial locations of the muscle or the muscles to evaluate, particularly determining the distribution of the vectors of the deformation and/or the synchronicity of the spatial evolution of the same;
c) outputting such sequences of spatial measurements in numeric and/or graphical format.

In essence the invention is based on the study of the fields of the deformations of one or more muscle structures through analysis of sequences of ultrasound images acquired during the period of time in which such structures perform a predefined action.

The images can be related to a single muscle element or two muscle elements (agonist-antagonist), or more muscular elements involved in single action. Where with the term image is to be intended both a traditional two-dimensional image (2D) and a three-dimensional volumetric acquisition (3D). The sequences of images are typically synchronous between them or temporally staggered by a known amount in order to allow the comparison between the measured deformation fields.

According to an embodiment, the method provides to evaluate the uniformity of muscle activity within same muscles and/or different muscle elements involved in the same action and/or synchronicity in the deformation between agonist and antagonist muscles through comparison of the measurements in terms of intensity and/or timing of events.

Particularly, it can be provided the step of comparing the behaviour in time of the deformation of an agonist with the behaviour of the deformation in the corresponding antagonist muscle to detect situations of overload of the former with reference to the latter, or vice versa, for example, by measuring the time shift between a contraction curve of the agonist muscle and a relaxation curve of the corresponding antagonist muscle.

The evaluation of the muscle deformation is advantageously performed through “Optical Flow” or Particle Image Velocimetry” techniques (OF-Ply) as disclosed, for example, in Singh A. Optic Flow Computation: A Unified Perspective. Piscataway, N.J.: IEEE Comput. Soc. Press, 1992, Barron J L, Fleet D J, Beauchemin S. Performance of optical flow techniques. International Journal of Computer Vision 1994;12:43-77, Adrian R J Twenty years of particle image velocimetry. Experiments in Fluids 2005; 39, 159-169. These methods of image analysis, known as “ speckle tracking ” or “feature tracking” in echocardiography, are widely used in cardiology to evaluate the performance of the heart muscle in his periodical contraction and relaxation as taught by the documents EP1520517 and U.S. Pat. No. 7,343,031.

Document EP2347716A1 discloses how the so-called Block Matching, a well-known PIV technique, can be used for evaluation of motion vector distribution in muscles. Horn and Schunk's optical flow algorithm is disclosed in Jun Shi et Al, Recognition of Finger Flexion from Ultrasound Image with Optical Flow: A Preliminary Study, Biomedical Engineering and Computer Science (ICBECS), 2010 Internation Conference on, IEEE, PISCATAWAY, N.J., USA, 23 Apr. 2010, pages 1-4, while the primal-dual optical flow algorithm is disclosed in Ji-Zhou Li et Al., Estimation of longitudinal muscle motion using primal-dual algorithm, Biomedical Engineering International Conference (BMEICON), 2012, IEEE, 5 December 2012, pages 1-5.

The innovative element in the present invention is a review of these methods and the consequent adaptation for the evaluation of the dynamic properties of the different regions with different muscle characteristics during a movement with a predefined, not sinusoidal, temporal profile as well as in the comparative assessment of different elements of different muscle groups.

Particular importance is posed to the evaluation of the deformation and, in particular, the strain rate of skeletal muscles' elements, and thus to the uniformity and balance of muscular activity within the same muscles, as well as in the synchronicity of the deformation between agonist and antagonist muscles. In the above mentioned references only motion vector distribution is determined, i.e. instantaneous information of the movement. Deformations/strain rates are not instantaneous quantities, but integral quantities that have to be derived from motion vectors. Further processing of motion vectors is envisaged, for example in EP2347716A1, but it is limited to the calculation of several type of scalars (eigenvalues, divergence, rotation) not useful for determining measurements of deformations and/or strain rates which represent the key of the invention.

To this end, the method according to the invention involves calculating not simply the velocity of points in the image sequence, but relative motion, thus tissue deformation, for example by calculating the spatial variation of such velocities along points related by their brightness in subsequent images of the sequence or sequences of input images.

According to a particularly advantageous embodiment, the method provides for calculating the main directions of the deformations and the deformation values in such directions and showing the input images together with the vectors of the deformations. Such vectors differ from the velocity vector because they represent, through the shape of arrows, the orientation along which the deformation occurs, the principal eigenvectors of the deformation tensor, possibly with length proportional to said values of deformation in said directions.

The herein described methodology, based on the use of sequences of muscle images (movies) in multiple locations at the same time, possibly combined with procedures for the execution of repeatable actions, allows the assessment of the muscular balance, which is considered a key element in the latest training, physiotherapic, and physical correction techniques.

According to another aspect, the invention relates to a device for actuating the above method comprising:
a) an input for receiving one or more sequences of two-dimensional or three-dimensional echographic images of the muscle or the muscles under investigation;
b) a processing unit for transforming such sequence or sequences of images in sequences of spatial measurements;
c) a processing unit;
d) an output for outputting such sequences of spatial measurements in numeric and/or graphical format.

The processing unit is configured to transform such sequence or sequences of images in sequences of measurements of deformations and/or strain rates in more spatial locations of the muscle or the muscles to evaluate performing one or more steps of the method according to the invention, particularly to determine the distribution of the vectors of the deformation and/or the synchronicity of the spatial evolution of the same.

Advantageously, the processing unit may be configured to evaluate the uniformity of muscle activity within same muscles and/or different muscle elements involved in the same action and/or the synchronicity of the deformation between agonist and antagonist muscles through comparison of the measurements in terms of intensity and/or synchronicity. Particularly the processing unit can be configured to calculate the main directions of the deformations and the deformation values in said directions. In this case the device can advantageously output the sequence or sequences of images together with the vectors of deformations, for example superimposing said vectors on the input images. The vectors typically have the shape of arrows oriented in the principal directions and length proportional to the values of deformation in said directions. This representation is particularly advantageous as it provides an immediate graphical indication of the behaviour of the deformations in time.

The device is typically provided for being interfaced with an echographic apparatus, the input sequences of images coming from said apparatus via data exchange on a mass memory or connection, wired or wireless, direct or through a network LAN, WAN or the Internet.

Advantageously the device can be provided for being interfaced, directly or through the echographic apparatus, with a system for the execution of a predefined, repeatable joint movement and/or a system for electro-induced stimulations capable of inducing predefined muscle contractions in one or more sites to allow acquisitions during reproducible movements for inter-individual and intra-individual comparisons at different times of training or rehabilitation.

Specifically the device can be provided in combination with an echographic apparatus having at least a probe for acquiring sequences of two-dimensional or three-dimensional echographic images of one or more muscles, there being provided means for synchronizing the acquisition in different zones of the same muscle or of different muscles.

The echographic apparatus can also be provided with at least two probes contemporaneously drivable to synchronously acquire sequences of two-dimensional or three-dimensional echographic images related to different muscles or different zones of the same muscle.

It is possible also to consider the combination of the device with at least two echographic apparatus having at least one probe for acquiring sequences of two-dimensional or three-dimensional echographic images of one or more muscles under investigation. In this case the sequences of images related to different muscles or different zones of the same muscle are acquired synchronously using means for synchronizing the acquisition of the two echographic apparatus, such as, for example, a protocol of communication of the two apparatus or a manual or automatic trigger, which, in its simplest configuration, is a switch manually actuated on both the apparatus.

According to a particularly advantageous embodiment, it is possible to consider the device in combination with a cinematically constrained machine, like those used in fitness or rehabilitation programmes as well as machines specifically used in other disciplines such as Gyrotonic or Pilates as well as GAIT analysis or with orthodontic appliances, and/or an electro-stimulation machine for muscle stimulation in coded and repeatable sequences. In this case the device may advantageously comprise an input for receiving such coded sequences through manual input or direct data exchange through an interface with the cinematically constrained machine or the electro-stimulation machine, the processing unit being sensitive to such input to perform comparisons between the deformations or strain rates as a function of the type of the stress induced by the machine to the muscle or muscles to evaluate.

According to another aspect, the invention relates to an echographic apparatus for evaluating the uniformity of the muscle activity within same muscles and/or different muscle elements involved in the same action and/or the synchronicity of deformation between agonist muscles and antagonist muscles. The apparatus comprises a device according to the invention capable to measure strain and/or strain rates from sequences obtained acquiring with one or more echographic probes one or more zones of the same muscle or of different muscles involved in the same action and operate a comparison between such measurements in terms of intensity and/or synchronicity.

According to an improvement, the apparatus may comprise at least two probes constrained among them for being placed on different zones of the same muscle and/or different muscles to contemporaneously acquire the related echographic images.

Advantageously the probe or the probes are supported by a device having a shape so as to be kept constrained with the anatomical element comprising the muscle to evaluate, such as, for example, a bandage, a collar, a brace or the like.

Further improvements of the invention will form the subject of the dependent claims.

The characteristics of the invention and the advantages derived therefrom will be more apparent from the following description of non-limiting embodiments, illustrated in the annexed drawings, in which:

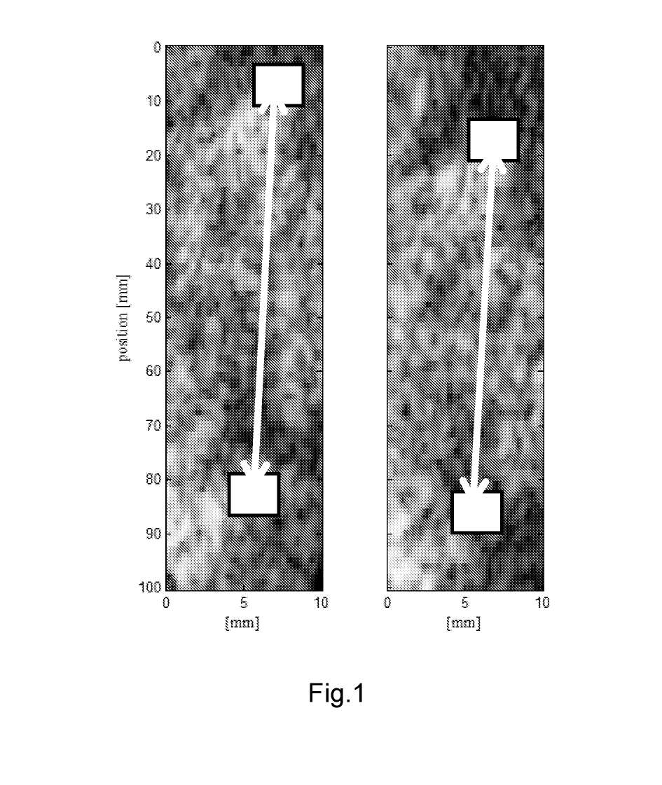

FIG. 1 shows an ultrasonic image of a muscle region (10×100 mm) at two instants during a muscle contraction. The arrow shows the muscle deformation through the relative displacement of two reference points recognizable in the two images and followed automatically in the sequence according to the method of the present invention;

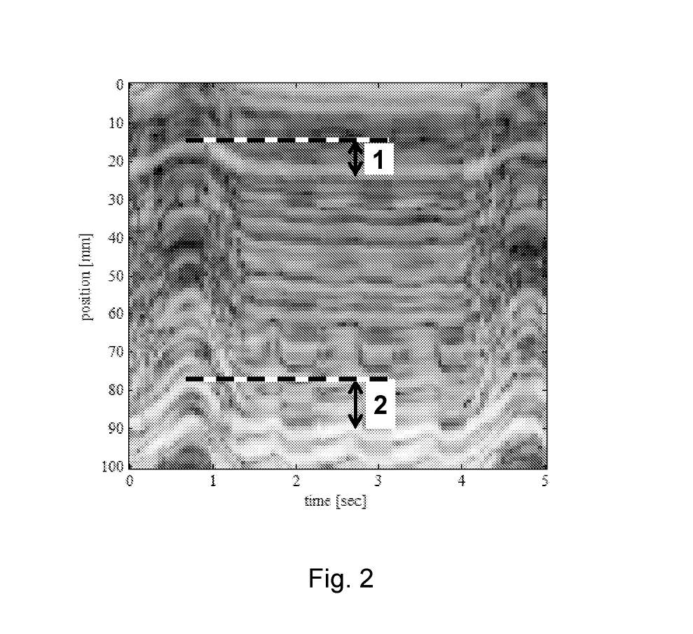

FIG. 2 shows a space-time ultrasound image (M-mode) along a muscle (10 cm) during the movement (5 seconds). This representation shows the spatial variation of the deformation moving from the proximal (1) to the distal region (2);

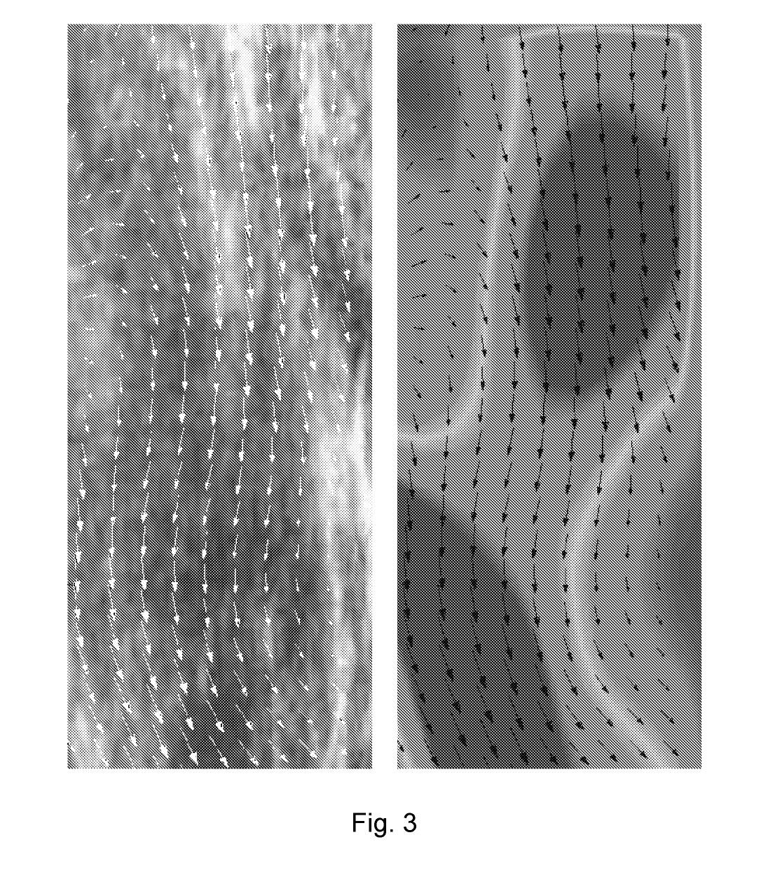

FIG. 3 shows an ultrasound image of a muscular tissue (left), and the evaluation of the strain rate field (indicated by the vectors) automatically calculated by the method and the device according to the invention. From this vector field is then possible to evaluate the properties of the deformation as shown by way of example in grayscale in the right image;

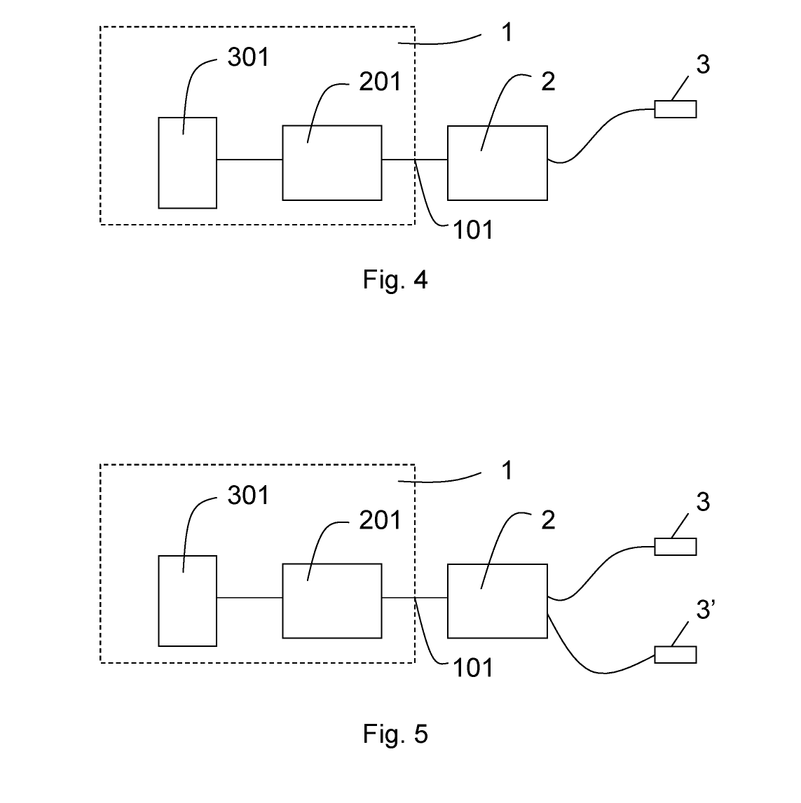

FIG. 4-7 illustrate an exemplified block diagram of some embodiments of the device according to the present invention.

The innovative technical element at the base of the present invention lends itself to multiple realizations of increasing complexity.

The common denominator of all these possible embodiments is represented by an image analysis device that, in its simplest form, is constituted by a processing unit capable of processing sequences of images generated by an ultrasound apparatus to provide sequences of measurements of spatial deformations such as those shown in FIGS. 1 and 2.

As already said, the objective evaluation of the muscle deformation can be advantageously performed with the already mentioned “Optical Flow” or “Particle Image Velocimetry” (OF-Ply) techniques adapted for the evaluation of dynamic properties of different regions with different muscular characteristics during a movement having a predetermined temporal profile, as well as the comparative evaluation of different elements of different muscle groups.

In essence, OF-PIV techniques are based on the concept of estimating motion of objects by comparing images taken at successive instants of time assuming that the brightness of each point of the original image moves rigidly in the images of the sequence. The images can be either two-dimensional three-dimensional. The following will, however, only deal with the two-dimensional case. Obviously, this should not be construed as limiting the scope of protection, but represents only an exemplification for an immediate grasp of the mathematical concepts used.

Indicating by B (x, y, t) the brightness of a point P of coordinates (x , y) at the time instant t, with Vx, Vy the velocity components, respectively along the x axis and along the y axis, it can be shown that the above assumption on B implies that the following equation should be satisfied:

$$\frac{\partial B}{\partial t} + {V}_{x} \frac{\partial B}{\partial x} + {V}_{y} \frac{\partial B}{\partial y} = 0$$

There are several ways to use this equation. The document Barron J L, Fleet D J, Beauchemin S. Performance of optical flow techniques, International Journal of Computer Vision 1994; 12:43-77 makes an overview of these possible ways of resolution.

Although any resolution technique may be employed, the inventors have found that in the particular case of the detection of deformations muscle via echo images, the techniques of windowing and error minimization are well appropriate in terms of complexity of calculation, and thus processing speed, and accuracy of the results.

Specifically, the speed for each point of the image can be advantageously estimated by defining a window of pixels W and minimizing the quantity:

$$E = {\sum }_{W} {( \frac{\partial B}{\partial t} + {V}_{x} \frac{\partial B}{\partial x} + {V}_{y} \frac{\partial B}{\partial y} )}^{2}$$

Posing the derivative to zero, i.e.

$$\{ \begin{matrix}\frac{\partial E}{\partial {V}_{x}} = 0 \\ \frac{\partial E}{\partial {V}_{y}} = 0\end{matrix}$$

We obtain

$$\{ \begin{matrix}{\sum }_{W} ( \frac{\partial B}{\partial t} + {V}_{x} \frac{\partial B}{\partial x} + {V}_{y} \frac{\partial B}{\partial y} ) \frac{\partial B}{\partial x} = 0 \\ {\sum }_{W} ( \frac{\partial B}{\partial t} + {V}_{x} \frac{\partial B}{\partial x} + {V}_{y} \frac{\partial B}{\partial y} ) \frac{\partial B}{\partial y} = 0\end{matrix}$$

That provides the following linear system

$$[ \begin{matrix}{\sum }_{W} \frac{\partial B}{\partial x} \frac{\partial B}{\partial x} {\sum }_{W} \frac{\partial B}{\partial x} \frac{\partial B}{\partial y} \\ \\ {\sum }_{W} \frac{\partial B}{\partial x} \frac{\partial B}{\partial y} {\sum }_{W} \frac{\partial B}{\partial y} \frac{\partial B}{\partial y}\end{matrix} ] \cdot [ \begin{matrix}{V}_{x} \\ \\ \\ {V}_{y}\end{matrix} ] = [ \begin{matrix}{\sum }_{W} \frac{\partial B}{\partial t} \frac{\partial B}{\partial x} \\ \\ {\sum }_{W} \frac{\partial B}{\partial t} \frac{\partial B}{\partial y}\end{matrix} ]$$

from which we can derive the unknowns Vx and Vy and, therefore, the field of velocities.

The deformation rates calculated by the method and the device according to an embodiment of the invention are represented by the changes of velocities in space i.e. by the gradient of the velocity vectors

$$∇ \overline{V} = [ \begin{matrix}\frac{\partial {V}_{x}}{\partial x} & \frac{\partial {V}_{x}}{\partial y} \\ & \\ \frac{\partial {V}_{y}}{\partial x} & \frac{\partial {V}_{y}}{\partial y}\end{matrix} ]$$

This matrix can be written as a sum of a symmetric matrix U and an antisymmetric matrix R in the following way

$$∇ \overline{V} = [ \begin{matrix}\frac{\partial {V}_{x}}{\partial x} & \frac{1}{2} ( \frac{\partial {V}_{x}}{\partial y} + \frac{\partial {V}_{y}}{\partial x} ) \\ & \\ \frac{1}{2} ( \frac{\partial {V}_{x}}{\partial y} + \frac{\partial {V}_{y}}{\partial x} ) & \frac{\partial {V}_{y}}{\partial y}\end{matrix} ] + [ \begin{matrix}0 & \frac{1}{2} ( \frac{\partial {V}_{x}}{\partial y} - \frac{\partial {V}_{y}}{\partial x} \\ & \\ - \frac{1}{2} ( \frac{\partial {V}_{x}}{\partial y} - \frac{\partial {V}_{y}}{\partial x} ) & 0\end{matrix} ]$$

The symmetric matrix U is a pure deformation rate tensor, while the asymmetric matrix R a pure rigid rotation rate involving no deformation by itself.

According to a particularly advantageous implementation form, the invention comprises the step to diagonalize the symmetric matrix to determine the lengthening/shortening (eigenvalues) along the principal directions (eigenvectors), i.e. the amount of pure deformation without shear. To such extent the method provides for calculating the principal directions of the deformations and the deformation values in such directions and showing the input images together with the vectors of the deformations. The vectors typically have the shape of arrows oriented in the principal directions and length proportional to the values of deformation in said directions as shown in FIG. 3.

This is particularly advantageous because it allows for an immediate feedback, also visual, of the behaviour of the deformations of the muscles under examination in terms vectors. It is, in fact, not so much the tissue velocity but the strain fields, i.e. the knowledge of the distribution of the deformation in the 2D or 3D space contained in the images, that allows to make direct and immediate comparisons between parts of the same muscle or of different muscles in order to evaluate the uniformity of the muscle action, as well as the synchrony of the deformation, aspects necessary to develop innovative methods of training or rehabilitation able to analytically evaluate individual progress and the answers to specific activities, and to balance the development of different muscle groups, reducing the risk of injury due to an imbalance between agonist and antagonist muscles or to an intensification of efforts in limited regions.

The image analysis device of the present invention lends itself to many embodiments.

In its simplest form shown in FIG. 4, the device 1 is provided in combination with an echographic apparatus 2 having a probe 3 capable of acquiring sequences of images. The apparatus 2 is connected in a physical manner or through wireless computer connections, to an input 101. It is also possible to provide that the exchange of data between apparatus 2 and device 1 is carried out through mass memories or in a way completely independent from the operation mode of the echographic apparatus 2 that, for this reason, can be of any type. The processing unit 201 of the device 1 reads the input sequences and process them to assess the spatial and temporal distribution of muscle deformation and show the results of the analysis on a monitor 301 in graphics and/or numeric form. The processing unit 201 may be a dedicated microprocessor system or, more generally, a PC also of the general purpose type. The characteristics of the unity 201 will obviously reflect on the processing speed.

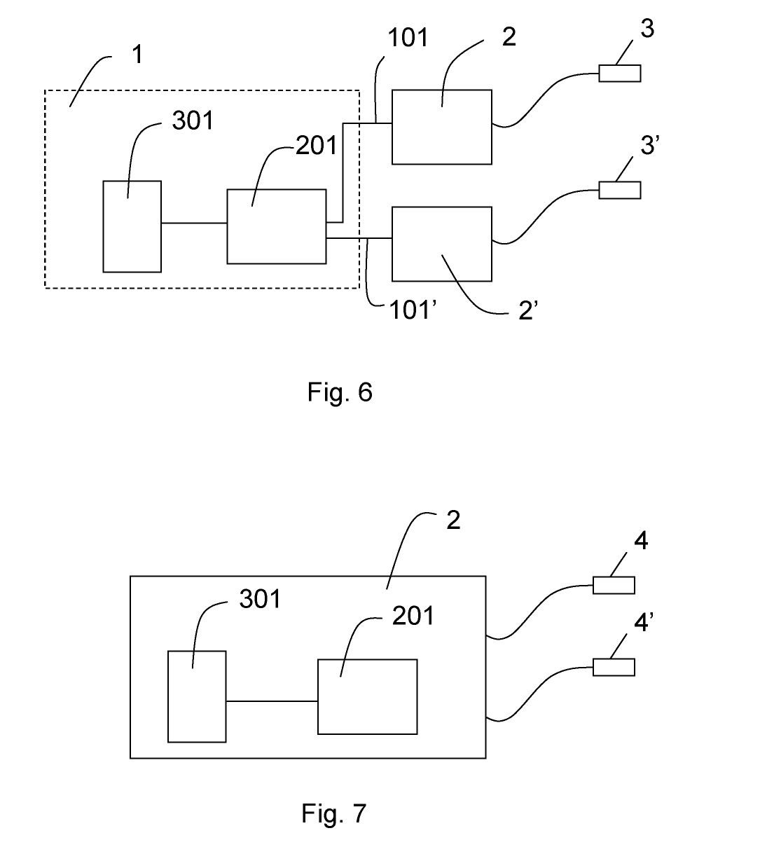

A more sophisticated embodiment, shown in FIG. 5, comprises two or more ultrasound probes 3, 3′ connected to the same echographic apparatus 2 for the simultaneous acquisition on two or more muscle groups so that the device 1 can perform a comparative evaluation of the relative deformations. In this case it is necessary that the apparatus used is capable of handling two or more probes simultaneously or multiplexed so as to ensure temporally staggered acquisitions of a known quantity. It is also possible to envisage that the device 1 is interfaced with two or more echographic apparatus 2, 2′ as shown in FIG. 6. This allows the use of conventional echographic apparatus, leaving, for example, to the user the responsibility for checking the acquisition times for example by simultaneously pressing the freeze of the two apparatus or by connecting in parallel a pedal that acts on the freeze key of each echographic apparatus.

This solution is definitely more flexible in terms of use of ultrasound systems, but certainly not the best in terms of effectiveness of the device.

The optimum solution is, in fact, the one that allows the integration of the device according to the invention within an echographic apparatus as schematically shown in FIG. 7. The device 1, in this case, may be part of the processing system of the ultrasound images with the monitor 301 that coincides with the same main monitor of the apparatus, thus creating a very compact system dedicated to the analysis of muscle deformations.

As far as ultrasound probes 4 are concerned, these can be of the traditional type or built ad hoc, for example to be integrated within a band to be placed around a leg for the analysis of the deformation of the leg muscles, or a collar or a bust for analyzing other anatomic regions.

According to a particularly advantageous implementation form, the device according to the invention is provided in combination with an auxiliary mechanical system to simplify the execution of predefined movements in a systematic and reproducible way, or to an electro-stimulation machine capable of inducing predetermined muscle contractions in one or multiple sites. In this way it is possible to perform reproducible acquisitions during reproducible movements for inter-individual and intra -individual comparisons at different times of training or rehabilitation. Such systems may be constrained kinematic machines like, for examples, those used in fitness or rehabilitation programmes, as well as specific machines of other disciplines such as, for example, the Gyrotonic or Pilates, or the equipment for GAIT analysis, or specific orthodontic appliances, and/or an electro-stimulation machine for muscle stimulation in coded and repeatable sequences.

In this case the device 1 may advantageously comprise an input (not shown in figures) for receiving such coded sequences through manual input or direct data exchange through an interface with the cinematically constrained machine or the electro-stimulation machine so that the processing unit 201 could perform comparisons between the deformations or strain rates as a function of the type of the stress induced by the machine to the muscle or muscles to evaluate.

1. Method for quantitative dynamic evaluation of skeletal muscles functionality, characterized in comprising the following steps:
a) receiving one or more sequences of two-dimensional or three-dimensional echographic images of the muscle under investigation;
b) transforming such sequence or sequences of images in sequences of measurements of deformations and/or strain rates in more spatial locations of the muscle or the muscles to evaluate;
c) outputting such sequences of spatial measurements in numeric and/or graphical format.

2. Method according to claim 1, wherein step b) comprises determining the distribution of the vectors of the deformation and/or the synchronicity of the spatial evolution of the same.

3. Method according to claim 1 or 2, wherein step a) comprises receiving sequences of images related to different muscles and/or different zones of the same muscle.

4. Method according to claim 3, wherein the sequences of images are substantially synchronous or temporally shifted of a known quantity.

5. Method according to claim 1, characterized in comprising the step of evaluating the uniformity of muscle activity within same muscles and/or different muscle elements involved in the same action and/or the synchronicity of the deformation between agonist and antagonist muscles through comparison of the measurements in terms of intensity and/or synchronicity.

6. Method according to claim 1, characterized in comprising the step of comparing the behaviour of the deformation of an agonist muscle with the behaviour of deformation of the correspondent antagonist muscle to detect situations of overload of the former with reference to the latter or viceversa.

7. Method according to claim 6, wherein a contraction curve of the agonist muscle and a relaxation curve of the corresponding antagonist muscle are shown, the situations of overload being detected by measuring the temporal shift between such curves.

8. Method according to claim 1, wherein step b) comprises determining the distribution of the deformation and/or strain rates through post-processing of Optical Flow and/or Particle Image Velocimetry techniques.

9. Method according to claim 1, characterized in comprising the step of calculating the main directions of the deformations and the deformation values in such directions and showing the input images together with the vectors of the deformations, such vectors having the shape of arrows oriented in such main directions and length proportional to said values of deformation in said directions.

10. Method according to claim 1, characterized in comprising the following steps:
calculating the velocity of points or zones having the same brightness in subsequent images of the input sequence or sequences of images;
calculating the spatial gradient matrix of said velocities;
dividing the spatial gradient matrix in a symmetrical matrix of pure deformation and in an asymmetrical matrix of pure rotation;
diagonalizing the symmetrical matrix with calculation of the related eigenvalues;
calculating the corresponding eigenvectors;
showing, overlapped on the input images, arrows oriented in the directions of the eigenvectors and with length proportional to the eigenvalues.

11. Device for quantitative dynamic evaluation of skeletal muscles functionality, characterized in comprising:
a) an input (101) for receiving one or more sequences of two-dimensional or three-dimensional echographic images of the muscle or the muscles under investigation;
b) a processing unit (201) for transforming such sequence or sequences of images in sequences of spatial measurements;
c) an output (301) for outputting such sequences of spatial measurements in numeric and/or graphical format,
characterized in that such processing unit (201) is configured to transform such sequence or sequences of images in sequences of measurements of deformations and/or strain rates in more spatial locations of the muscle or the muscles to evaluate performing one or more steps of the method according to claim 1.

12. Device according to claim 11, wherein said processing unit (201) is configured to determine the distribution of the vectors of the deformation and/or the synchronicity of the spatial evolution of the same.

13. Device according to claim 11, wherein the processing unit (201) is configured to evaluate the uniformity of muscle activity within same muscles and/or different muscle elements involved in the same action and/or the synchronicity of the deformation between agonist and antagonist muscles through comparison of the measurements in terms of intensity and/or synchronicity.

14. Device according to claim 11, wherein the processing unit (201) is configured to calculate the main directions of the deformations and the deformation values in said directions, the device outputting the sequence or sequences of images together with the vectors of deformations, such vectors having the shape of arrows oriented in such main directions and length proportional to said values of deformation in said directions.

15. Device according to claim 11, characterized in that it is provided for being interfaced with an echographic apparatus (2), the input sequences of images coming from said apparatus via data exchange on a mass memory or connection, wired or wireless, direct or through a network LAN, WAN or the Internet.

16. Device according to claim 11, characterized in that it is provided for being interfaced, directly or through the echographic apparatus (2), with a system for the execution of a predefined, repeatable joint movement and/or a system for electro-induced stimulations capable of inducing predefined muscle contractions in one or more sites.

17. Device according to claim 11, characterized in that it is provided in combination with an echographic apparatus (2) having at least a probe (3) for acquiring sequences of two-dimensional or three-dimensional echographic images of one or more muscles, there being provided means for synchronizing the acquisition in different zones of the same muscle or of different muscles.

18. Device according to claim 11, characterized in that it is provided in combination with an echographic apparatus (2) having at least two probes (3, 3′) contemporaneously drivable to synchronously acquire sequences of two-dimensional or three-dimensional echographic images related to different muscles or different zones of the same muscle.

19. Device according to claim 11, characterized in that it is provided in combination with at least two echographic apparatus (2, 2′) having at least one probe (3, 3′) for acquiring sequences of two-dimensional or three-dimensional echographic images of one or more muscles under investigation, there being provided means for synchronizing the acquisition of the two echographic apparatus to allow sequences of images related to different muscles or different zones of the same muscle to be acquired synchronously.

20. Device according to claim 11, characterized in that it is provided in combination with a cinematically constrained machine and/or a machine for muscular activity and/or an electro-stimulation machine for muscle stimulation in coded and repeatable sequences, the device comprising an input for receiving such coded sequences through manual input or direct data exchange through an interface with the machine, the processing unit being sensitive to such input to perform comparisons between the deformations or strain rates as a function of the type of the stress induced by the machine to the muscle or muscles to evaluate.

21. Echographic apparatus for evaluating the uniformity of the muscle activity within same muscles and/or different muscle elements involved in the same action and/or the synchronicity of deformation between agonist and antagonist muscles, characterized in comprising a device (1) according to claim 11 capable to measure deformations and/or strain rates from sequences obtained acquiring with one or more echographic probes (3) one or more zones of the same muscle or of different muscles involved in the same action and operate a comparison between such measurements in terms of intensity and/or synchronicity.

22. Echographic apparatus according to claim 21, characterized in comprising at least two probes (3, 3′) constrained among them for being placed on different zones of the same muscle and/or different muscles to contemporaneously acquire the related echographic images.

23. Echographic apparatus according to claim 21, characterized in that the probe or the probes (3, 3′) are supported by a device having a shape so as to be kept constrained with the anatomical element comprising the muscle to evaluate.

24. Echographic apparatus according to claim 23, characterized in that such device has the shape of a bandage, a collar, a brace or the like.
